(19) United States
(12) Patent Application Publication
(10) Pub. No.: US 2026/0308623 A1
(43) Pub. Date: Oct. 8, 2026

(54) SINGLE PLASMID SYSTEM FOR AAV PRODUCTION

(71) Applicant: Solid Biosciences Inc., Charlestown, MA (US)

(72) Inventor: Sarath Chandra Mandava, Malden, MA (US)

(21) Appl. No.: 19/482,265

(22) PCT Filed: May 17, 2024
(86) PCT No.: PCT/US2024/029907

§ 371 Date: Nov. 7, 2025

Related U.S. Application Data
(60) Provisional application No. 63/467,584, filed on May 18, 2023.

(51) Int. Cl. C12N 15/86

(52) U.S. Cl. CPC C12N 15/86; C12N 2750/14122; C12N 2750/14143; C12N 2750/14152

(57) ABSTRACT

The invention described herein provides an improved system and method for rAAV production. The system of the invention includes a single recombinant DNA vector that comprises a gene of interest (GO I) to be delivered to a target cell, the Rep and Cap coding sequences, as well as the Helper gene coding sequences for rAAV packaging.

### REFERENCE TO RELATED APPLICATION

This application claims priority to and the benefit of the filing date of U.S. Provisional Patent Application No. 63/467,584, filed on May 18, 2023, the entire contents of which are incorporated herein by reference.

### BACKGROUND OF THE INVENTION

In the early days of rAAV production, cultured cells are first infected with adenovirus, then transfected with two additional DNA plasmids: one plasmid containing a pair of essential rAAV genes (e.g., Rep/Cap genes), and the other plasmid carrying a sequence of a gene of interest (GOI). This method produces a reasonable yield of rAAV particles, but there is a risk of adenovirus contaminating the preparation which can put patients treated with rAAV at risk.

In 1998, different research groups determined that the key genes of the adenovirus can be shifted into a third “helper” plasmid, and all three plasmids can be transfected into a preferred host cell at the same time to produce rAAV particles, circumventing the potential contamination of rAAV. Since then, this method known as the “triple transfecfion” technique has been widely used as the main method to produce recombinant adeno associated virus (rAAV), including commercial manufacturing of AAV viral stocks for gene therapy.

However, a persistent problem of using AAV-based gene therapy is the demand for large quantities of suitable AAV viral particles (e.g., in the range of about 1×10¹⁴ to 1×10¹⁵ vector genome) for efficacious treatment.

Today, rAAV is considered a key platform to deliver numerous types of genes for gene therapy, and the number of rAAV-based therapies in development continues to increase. Therefore, there is a need for further improving large scale rAAV production.

### SUMMARY OF THE INVENTION

Exemplary embodiments of the invention described herein are provided here as numbered paragraphs.

1. A recombinant DNA vector comprising:
(a) a gene-of-interest (GOI) flanked by a 5′ adeno-associated virus (AAV) ITR (such as AAV2 5′ ITR) sequence and a 3′ ITR sequence (such as AAV2 3′ ITR);
(b) a coding sequence for an AAV Rep (such as AAV2 Rep, or Rep2) compatible with said 5′ AAV ITR and/or said 3′ AAV ITR;
(c) a coding sequence for an AAV Cap; and
(d) a coding sequence for AAV Helper genes sufficient for enabling AAV packaging;
wherein the coding sequence for the AAV Rep and the coding sequence for the AAV Cap are within a RepCap cassette comprising an operably-linked RepCap promoter (such as the AAV P5 promoter); and,
wherein the GOI flanked by the 5′ and 3′ ITRs is positioned upstream of (e.g., adjacent to, or immediately 5′ to) the 5′ end of the promoter of the RepCap cassette.

2. The recombinant DNA vector of paragraph 1, wherein expression of said AAV Rep, Cap and Helper genes in a host cell comprising AAV helper genes is sufficient to package an AAV vector genome (vg) comprising the GOI flanked by the 5′- and 3-ITR sequences into an AAV capsid comprising said AAV Cap.

3. The recombinant vector of any one of paragraphs 1-2, wherein the recombinant vector is in a plasmid.

4. The recombinant vector of any of paragraphs 1-3, wherein the GOI is within a pro-AAV cassette comprising the GOI operably linked to a promoter.

5. The vector of paragraph 4, wherein the pro-AAV cassette further comprises:
(1) an enhancer that promotes the transcription of the GOI from the promoter;
(2) a 5′ UTR;
(3) a Kozak sequence;
(4) a heterologous intron that promotes transcription and/or translation of the GOI;
(5) a 3′ UTR;
(6) a WPRE sequence; and/or
(7) a polyA signal sequence.

6. The recombinant vector of any one of paragraphs 4-5, wherein the RepCap cassette and the pro-AAV cassette:
i) are immediately adjacent to each other (e.g., with substantially no intervening polynucleotide sequence);
ii) are not immediately adjacent to each other;
iii) have the same transcription direction;
iv) having opposite transcription direction.

7. The recombinant vector of paragraph 6, further comprising a bacterial replication Ori gene, a selection marker (such as an antibiotic resistance gene, e.g., Kan^{R} or Amp^{R}) under the transcriptional control of a selection marker promoter.

8. The recombinant vector of any one of paragraphs 1-7, wherein said GOI is a functional equivalent of dystrophin (e.g., a dystrophin minigene encoding a functional micro-dystrophin protein).

9. The recombinant vector of any one of paragraphs 1-8, wherein said GOI includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

10. The recombinant vector of any one of paragraphs 1-8, wherein the GOI is a microdystrophin gene (e.g., one described in U.S. Pat. Nos. 7,906,111; 7,001,761; 7,510,867; 6,869,777; 8,501,920; 7,892,824; PCT/US2016/013733; or U.S. Pat. No. 10,166,272).

11. The recombinant vector of paragraph 10, wherein the microdystrophin gene comprises a coding sequence for R16 and R17 spectrin-like repeats for the full length dystrophin protein (such as one described in U.S. Pat. No. 7,892,824).

12. The recombinant vector of paragraph 11, wherein the microdystrophin gene comprises a coding sequence for the R1, R16, R17, R23, and R24 spectrin-like repeats of the full-length dystrophin protein (such as the microdystrophin gene described in PCT/US2016/013733).

13. The recombinant vector of any one of paragraphs 1-12, wherein said 5′ and 3′ AAV ITR sequences flanking said GOI are both from AAV1, AAV2, AAV3A, AAV3B, AAV4, AAV5, AAV6, AAV7, AAV8, AAV9, AAV10, AAV11, AAV12, AAV13, or AAV-DJ.

14. The recombinant vector of paragraph 13, wherein the tropism of the AAV include skeletal muscle (such as AAV1, AAV6, AAV7, AAV8, AAV9, or a derivative thereof, preferably AAV9 or a derivative thereof such as SLB-101 described in WO 2021/072197).

15. The recombinant vector of any one of paragraphs 1-14, wherein said AAV ITR, said AAV Rep, and said AAV Cap are from the same or different AAVs.

16. The recombinant vector of paragraph 15, wherein said AAV ITR is AAV2 ITR, said AAV Rep is Rep2 from AAV2, and said AAV Cap is Cap9 from AAV9 or a derivative thereof (such as SLB-101 described in WO 2021/072197).

17. The recombinant vector of any one of paragraphs 1-16, wherein said coding sequence for AAV Rep and Cap proteins is under the transcriptional control of a promoter, such as an AAV p5 promoter, an upstream HSV promoter, a modified p5 promoter lacking RBE (Rep-Binding Element), or a ubiquitous promoter (such as CMV promoter, EF1a promoter, CAG promoter, CB promoter etc).

18. The recombinant vector of paragraph 16, wherein the AAV RepCap promoter is an AAV p5 promoter.

19. The recombinant vector of any one of paragraphs 1-18, wherein said AAV helper genes comprise adenoviral, herpesviral, or papillomaviral genes for AAV packaging (such as E1A, E1B, E2A, E4 and VA RNA), optionally operably linked to a promoter as one transcriptional unit.

20. A plasmid comprising the recombinant vector of any one of paragraphs 1-19.

21. A composition or a kit, comprising the vector of any one of paragraphs 1-19 or the plasmid of paragraph 20.

22. A host cell comprising the recombinant vector of any one of paragraphs 1-19, or the plasmid system of paragraph 21.

23. The host cell of paragraph 22, which is a HEK 293 cell (such as an Expi293F cell), a HeLa cell, an A549 cell, a BHK cell, a VP2 cell, or an insect cell (such as Sf9).

24. A method of propagating/amplifying/producing a recombinant replication-defective AAV viral particle encapsidating the GOI of any one of paragraphs 1-19, the method comprising: introducing the vector of any one of paragraphs 1-19 or the plasmid of paragraph 20 into a host cell.

25. The method of paragraph 24, further comprising harvesting the recombinant replication-defective AAV viral particle from the host cell.

26. The method of paragraph 24 or 25, wherein the host cell is a HEK 293 cell (such as an Expi293F cell), a HeLa cell, an A549 cell, a BHK cell, a VP2 cell or an insect cell (such as Sf9).

27. The method of any one of paragraphs 24-26, wherein the recombinant vector or plasmid is introduced to the host cell by transient transfection.

It should be understood that any one embodiment of the invention described herein, including those described only in the examples or claims, can be combined with any one or more additional embodiments of the invention, unless expressly disclaimed or is improper.

### BRIEF DESCRIPTION OF THE DRAWINGS

FIGS. 1A and 1B each show a plasmid map of an exemplary plasmid to produce AAV viral particles, wherein the gene of interest (GOI) is a variant of microdystrophin as a non-limiting example of the GOI. μDys: microdystrophin.

FIGS. 2A and 2B show results of rAAV production using VP2 cells by transfection of the subject single plasmid systems compared to a dual plasmid system. rAAV titres were determined 1, 2 and 3 days post transfection (DPT). DT: dual transfection; OPT: one plasmid transfection with the Helper genes oriented with the same direction of transcription as the Rep/Cap genes, as depicted in FIGS. 1A and 1B; OPT-rev help: one plasmid transfection with the Helper gene oriented with a reverse direction of transcription compared to the Rep/Cap genes.

### DETAILED DESCRIPTION OF THE INVENTION

### 1. Overview

Conventional AAV production system utilizes three plasmid vectors (the so-called triple transfection vector system), one with a gene of interest (GOI) flanked by AAV ITR sequences, for packaging into the AAV viral particles; another with an expression cassette encoding the AAV Rep and Cap proteins useful for AAV packaging; and yet another provides the useful helper genes from other viruses (such as adenovirus, herpesvirus, or papillomavirus) for productive AAV life cycle.

The single transfection vectors of the invention described herein improve the conventional system by inserting both the GOI cassette, the Rep-Cap expression cassette and Helper expression cassette into a single plasmid, to create the subject single transfection vector (e.g., plasmid) for use in producing AAV viral particles.

It was surprisingly found that, despite the large size, transfection of singe plasmids comprising both the GOI cassette, the Rep-Cap expression cassette and Helper expression cassette efficiently produce AAV viral particles.

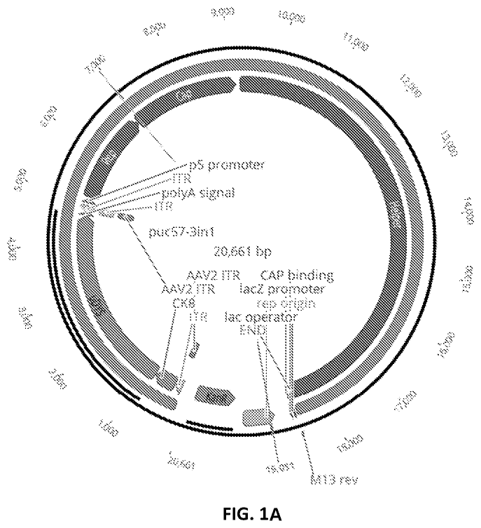

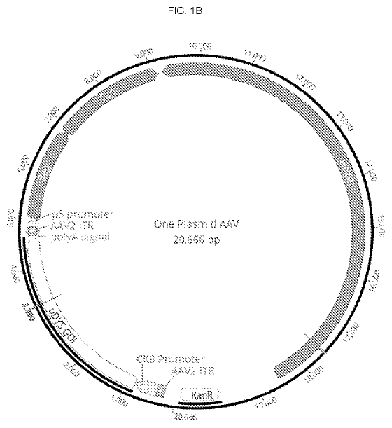

Further, in some embodiments, AAV production using the subject recombinant DNA vector (rDNA vector) results in reduced frequency of mispackaging. An exemplary such embodiment for the subject recombinant DNA vector comprises a configuration as shown in FIGS. 1A and 1B.

Thus, one aspect of the invention provides a recombinant DNA vector comprising (a) a gene-of-interest(GOI) flanked by a 5′ adeno-associated virus (AAV) ITR (such as AAV2 5′ ITR) sequence and a 3′ ITR sequence (such as AAV2 3′ ITR); (b) a coding sequence for an AAV Rep (such as AAV2 Rep, or Rep2) compatible with said 5′ AAV ITR and said 3′ AAV ITR; (c) a coding sequence for an AAV Cap; and (d) a coding sequence for AAV Helper genes sufficient for enabling AAV packaging; wherein the coding sequence for the AAV Rep and the coding sequence for the AAV Cap are within a RepCap cassette comprising an operably-linked RepCap promoter (such as the AAV P5 promoter); and wherein the GOI flanked by the 5′ and 3′ ITRs is positioned upstream of (e.g., adjacent to, or immediately 5′ to) the 5′end of the promoter of the RepCap cassette (with the transcription direction of the RepCap cassette as reference).

In certain embodiments, expression of said AAV Rep, Cap and Helper genes in a host cell comprising AAV helper genes is sufficient to package an AAV vector genome (vg) comprising the GOI flanked by the 5′- and 3-ITR sequences into an AAV capsid comprising said AAV Cap.

In certain embodiments, the vector or recombinant DNA vector is a plasmid.

In certain embodiments, the recombinant DNA vector is a plasmid, which may be suitable for transfection into an AAV packaging cell line, such as Expi, HEK293 (such as HEK293T cells), or other commonly used AAV packaging cell lines.

In certain embodiments, the GOI is within a pro-AAV cassette comprising the GOI operably linked to a promoter.

In yet another aspect, the invention provides a single plasmid system comprising the recombinant DNA vector disclosed herein.

As used herein, the term “plasmid” includes a nucleic acid molecule that can replicate independently of a cell chromosome. The term “plasmid” is intended to include circular nucleic acid molecules and linear nucleic acid molecules. Furthermore, the term “plasmid” is intended to include bacterial plasmids, cosmids, minicircles (Nehlsen et al., Gene Ther. Mol. Biol., 10:233-244, 2006; and Kay et al., Nature Biotechnology, 28:1287-1289, 2010) and ministrings (Nafissi et al., Mol Ther Nucleic Acids, 3:el65, 2014). In certain embodiments, the plasmid is a circular nucleic acid (DNA) molecule. In certain embodiments, the plasmid is a nucleic acid molecule that is of bacterial origin.

In some embodiments, the GOI is within a pro-AAV cassette comprising the GOI operably linked to a promoter.

In certain embodiments, the rDNA vector comprises two or more copies of the pro-AAV cassette. In certain embodiments, all copies of the pro-AAV cassette comprise the same GOI. In certain embodiments, at least two of the pro-AAV cassettes comprise different GOI. The latter embodiment can be useful, for example, if AAV vectors are used to deliver different parts of the same functional assembly, such as a CRISPR/Cas effector enzyme and the coding sequence for one or more guide RNAs.

In certain embodiments, the pro-AAV cassette further comprises: (1) an enhancer that promotes the transcription of the GOI from the promoter; (2) a 5′ UTR; (3) a Kozak sequence; (4) a heterologous intron that promotes transcription and/or translation of the GOI; (5) a 3′ UTR; (6) a WPRE sequence; and/or (7) a polyA signal sequence.

In some embodiments, the coding sequence for the AAV Rep and the coding sequence for the AAV Cap are within a RepCap cassette comprising an operably-linked RepCap promoter. In certain embodiments, the operably-linked RepCap promoter comprises the AAV P5 promoter.

In some embodiments, the RepCap cassette and the pro-AAV cassette are immediately adjacent to each other (e.g., with substantially no intervening polynucleotide sequence).

In some other embodiments, the RepCap cassette and the pro-AAV cassette are not immediately adjacent to each other.

In some embodiments, the RepCap cassette and the pro-AAV cassette have the same transcription direction.

In some other embodiments, the RepCap cassette and the pro-AAV cassette have opposite transcription direction.

In some embodiments, the pro-AAV cassette is upstream of the RepCap cassette.

In some embodiments, the GOI is in front of the coding sequence for the AAV Rep. In some embodiments, the GOI is in front of the coding sequence for the AAV Cap. In some embodiments, the GOI is in front of the coding sequences for both the AAV Rep and AAV Cap.

In some embodiments, the expression of the AAV Rep in a host cell comprising AAV helper genes is sufficient to package an AAV vector genome (vg) comprising the GOI flanked by the 5′- and 3-ITR sequences into an AAV capsid comprising the AAV Cap.

In some embodiments, the recombinant DNA vector (single transfection vector) further comprises a bacterial replication Ori gene, a selection marker (such as an antibiotic resistance gene, e.g., KanR or AmpR) under the transcriptional control of a selection marker promoter.

In certain embodiments, the GOI is a functional equivalent of dystrophin (e.g., a dystrophin minigene encoding a functional micro-dystrophin protein).

In certain embodiments, the GOI includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

Duchenne is a genetic muscle-wasting disease predominantly affecting boys, with symptoms that usually manifest between three and five years of age. Duchenne is a progressive, irreversible, and ultimately fatal disease that affects approximately one in every 3,500 to 5,000 live male births and has an estimated prevalence of 5,000 to 15,000 cases in the United States alone. Duchenne is caused by mutations in the dystrophin gene, which results in the absence or near-absence of dystrophin protein. Dystrophin protein works to strengthen muscle fibers and protect them from daily wear and tear. Dystrophin protein also serves as the cornerstone of the dystrophin glycoprotein complex, or DGC, a group of proteins that links the inner and outer components of muscle cells to ensure proper muscle function. Without functioning dystrophin and DGC, muscles suffer excessive damage from normal daily activities and are unable to regenerate, leading to the build-up of fibrotic, or scar, and fat tissue. More than 1,000 dystrophin gene mutations, which can be inherited or can occur spontaneously, have been identified in people with Duchenne. By their early teens, Duchenne patients typically lose their ability to walk and become dependent on a wheelchair for mobility. By their 20s, patients essentially become paralyzed from the neck down and require a ventilator to breathe. Though disease severity and life expectancy vary, a patient's quality of life dramatically decreases over time, with death typically occurring by early adulthood from either cardiac or respiratory complications.

There is no cure for Duchenne, and for the vast majority of patients, there are no satisfactory symptomatic or disease-modifying treatments. Glucocorticoid treatment, the current standard-of-care, has been shown to temporarily improve muscle strength, prolong the period of ambulation and slow the progression of Duchenne. However, glucocorticoid use is associated with well-known adverse side effects, including: severe weight gain, stunted growth, weakening of bone structure and metabolic dysfunctions, among others. The most commonly used glucocorticoids include prednisone and deflazacort (EMFLAZA).

In certain embodiments, the GOI comprises a synthetic dystrophin transgene construct, encoding a microdystrophin, under the transcriptinal control of a muscle specific promoter. In certain embodiments, the microdystrophin, when administered systemically, produces functional microdystrophin protein expression that not only stabilizes muscle membranes and protects muscle against injury, but also simultaneously restores the localization of DGC to the muscle membrane, notably increasing neuronal Nitric Oxide Synthase, or nNOS, concentration. In certain embodiments, the nNOS-restoring microdystrophins are more effective in improving muscle function and resistance to fatigue.

In certain embodiments, the GOI is a microdystrophin gene (e.g., one described in U.S. Pat. Nos. 7,906,111; 7,001,761; 7,510,867; 6,869,777; 8,501,920; 7,892,824; PCT/US2016/013733; or U.S. Pat. No. 10,166,272).

In certain embodiments, the microdystrophin gene comprises a coding sequence for R16 and R17 spectrin-like repeats for the full length dystrophin protein (such as one described in U.S. Pat. No. 7,892,824).

In certain embodiments, the microdystrophin gene comprises a coding sequence for the R1, R16, R17, R23, and R24 spectrin-like repeats of the full-length dystrophin protein (such as the microdystrophin gene described in PCT/US2016/013733).

In certain embodiments, the promoter driving the expression of the microdystrophin is a synthetic muscle-specific promoter (e.g., the CK8 promoter), which is derived from the naturally occurring muscle creatine kinase promoter, and is capable of driving expression of the microdystrophin transgene in skeletal, diaphragm and cardiac muscle tissues. In preclinical studies in small and large animal models, CK8 restricted microdystrophin transgene expression to these muscles.

In certain embodiments, the cap encodes a synthetic AAV capsid—AAV-SLB101 (see WO 2021/072197, incorporated herein by reference)—with enhanced muscle tropism and reduced liver uptake, which is able to more selectively deliver the drug to target tissue. The mdx mice dosed with the novel capsid showed increased biodistribution (vector genome copies) in representative muscle tissues and increased microdystrophin expression compared to those administered the AAV9 vector. In addition, there were lower vector genome copies in the liver compared to AAV9-administered animals, with the data supporting a preferential distribution of the novel capsid towards muscle tissue and away from the liver.

In certain embodiments, the GOI includes a gene useful for treating a cardiac disease (genetic cardiac disease), such as cardiomyopathy (e.g., BAG3).

Genetic cardiac disease, or inherited cardiac conditions, is an umbrella term to describe cardiac diseases caused by mutations in one or more genes.

Cardiomyopathy is a disease of the heart muscle that impairs the ability of the heart to pump blood to the rest of the body, resulting in arrhythmias, backup of blood into the lungs and other parts of the body, and ultimately heart failure. Forms of cardiomyopathy include dilated, or DCM, hypertrophic, or HCM, and arrhythmogenic cardiomyopathy.

DCM is characterized by left ventricular enlargement and systolic dysfunction leading to heart failure and/or arrhythmia with significantly heightened risk of sudden cardiac death. DCM is the most common indication for heart transplant and the third most common cause of heart failure, with an annual incidence rate of sudden cardiac death of between 2% to 4%. Approximately 20% to 37% of DCM is estimated to be based on genetic causes. Individual patient prognosis varies, but patients with certain mutation profiles often present with arrhythmia and/or heart failure by approximately age 40 and have an overall severe risk of adverse cardiac events. There is a significant unmet need for gene therapy in DCM given a lack of available disease-modifying therapies and a high morbidity/mortality with burdensome standard-of-care for these patients.

BAG3-mediated DCM is a rare cardiac disease and is characterized by mutations in the BAG3 gene. The BAG3 gene codes for the BCL-2-associated athanogene 3, or BAG3, protein. Sufficient levels of functional BAG3 are required for healthy cardiac function. BAG3 gene mutations lead to reduced BAG3 protein levels and ultimately DCM. Deletions and truncations in the BAG3 protein that result in haplo-insufficiency have been associated with the development of dilated cardiomyopathy resulting from myofilament damage, poor contraction, left ventricular dysfunction, dilatation and heart failure. Mechanistically, these physiologic impairments are caused from decreased BAG3 protein levels leading to heat shock protein dysfunction and a subsequent build-up of mis-folded, dysfunctional proteins in the sarcomere, which results in poor sarcomere integrity, increased mechanical stress, inflammation, remodeling and fibrosis. BAG 3-mediated DCM accounts for approximately 3-4% of all diagnosed cases of DCM, representing a prevalent population of about 29,000 patients in the United States. The most common presentation of BAG3-mediated DCM is dyspnea but can range from leg swelling and fatigue to more severe complications including thromboembolic events, arrhythmias or even sudden cardiac death. Thus, activities of daily life are severely impacted in patients with BAG3-mediated DCM. Once patients are symptomatic, mortality is approximately 25% at one year and approximately 50% at five years. The penetrance of DCM in patients with BAG 3 haploinsufficiency is 80% by age 40.There are no approved therapies to address the underlying cause of the disease and the current standard of care relies on symptomatic treatment depending on disease severity/progression. Treating the underlying cause of BAG3-mediated DCM requires expression of functional BAG3 within the heart muscle only.

In certain embodiments, the GOI includes a gene useful for treating a genetic cardiac disease, such as cardiomyopathy, including BAG3-mediated DCM. In certain embodiments, the GOI comprises a coding sequence for BAG3.

In certain embodiments, the GOI comprises a transgene encoding full length wild-type BAG3 protein. In certain embodiments, expression of the transgene is driven by a muscle-specific promoter. In certain embodiments, the cap encodes an AAVrh74 capsid or a derivative thereof having similar tropism.

Preclinical data in wild type mice indicate that the AAVrh74 capsid and muscle-specific promoter combination show enhanced cardiac biodistribution and expression and decreased liver expression relative to an AAV9 capsid and constitutive promoter combination at doses of 5E13 vg/kg or less.

In certain embodiments, the GOI includes a gene useful for treating a neuromuscular disease, such as Friedreich's ataxia (e.g., FXN). In certain embodiments, the GOI comprises a coding sequence for FXN.

Friedreich's ataxia or “FA” is a rare, inherited, multisystem, genetic disease caused by loss of functional frataxin protein, or FXN. One in every approximately 40,000 to 50,000 people suffer from FA, with a carrier rate between 1:60 and 1:100, making FA the most common hereditary ataxia. The average age of diagnosis is in the early teens, which leads to many undiagnosed patients. Males and females are equally affected by FA. Approximately 9,000 patients in the United States and approximately 26,000 patients in the European Union are affected by FA.

FA is a multisystem disease having both neurological and cardiac manifestations. FA affects the nerves and spinal cord, causing loss of control of body movements (ataxia). The most common manifestations of FA are progressive neurological symptoms, including loss of balance and coordination, loss of sensation in the arms and legs and loss of vision and hearing. A patient with FA will usually need a wheelchair within ten to twenty years of symptom onset, may be completely incapacitated in later stages of the disease and may have a shortened life span. Mortality in FA is most commonly due to cardiac complications as 59% of FA deaths are from cardiac dysfunction. Presently, only symptomatic treatment options are available for FA, with none addressing the underlying cause of the disease, the defective frataxin gene.

The primary cause of FA is a triplet repeat mutation in the frataxin gene, which encodes for FXN, a mitochondrial iron-binding protein involved in iron homeostasis. FA is an autosomal recessive disorder caused by a defect in both copies of the frataxin gene, most commonly due to GAA repeat expansions. Normal frataxin has GAA repeat levels ranging from 1 to 43. Mutated frataxin genes have GAA repeat levels ranging from 44 to 1,700. An increased number of GAA repeats between exon 1 and 2 of the frataxin gene results in abnormally low levels of the frataxin protein and accumulation of intracellular iron. This excess of iron in the heart and brain cells of FA patients promotes the production of toxic reactive oxygen species and leads to mitochondrial damage. This damage ultimately leads to progressive nervous system degeneration, movement problems, and cardiac dysfunction. Disease severity is correlated with increasing GAA repeat number in the frataxin gene.

Mutations in both copies of the frataxin gene are the cause of the disease. Onset and progression of the disease are directly related to the amount of FXN expressed in cells. Carriers of one normal and one mutated copy of the frataxin gene are clinically normal. All patients express some level of FXN, and thus will not mount an immune response to the therapeutic protein. Proof-of-concept data in animal models suggests that gene therapy may be a viable treatment for FA. Thus, delivery of gene therapy to one or more impacted tissues may provide clinical benefit to patients.

In certain embodiments, the GOI comprises a transgene encoding full length wild-type FXN protein. In certain embodiments, expression of the transgene is driven by a universal promoter capable of driving expression of the encoded FXN in target tissues of FA, such as those of the central nervous system and heart. In certain embodiments, the cap encodes an AAV9 capsid or a derivative thereof having similar tropism. In certain embodiments, the AAV viral particle comprising the GOI, as produced by the method and composition of the invention, is administered intravenously, intrathecally, or both. In certain embodiments, the AAV viral particle is administered intravenously and intrathecally to target both the neurological and cardiac impairments experienced by patients, in order to more comprehensively target disease pathology.

A similar AAV viral particle produced via an HSV-based manufacturing process (e.g., a transgene encoding full length FXN protein packaged into an AAV9 capsid and under control of a universal promoter intended to drive expression of FXN in target tissues of disease, especially those of the central nervous system and heart), has shown in preclinical studies in mouse (and NHP) to enhance survival and cardiac function in a cardiac-specific FXN knockout mouse, and an overall favorable safety profile at high FXN expression levels, thus providing preclinical proof of concept of the gene therapy.

In certain embodiments, the 5′ and 3′ AAV ITR sequences flanking said GOI are both from AAV1, AAV2, AAV3A, AAV3B, AAV4, AAV5, AAV6, AAV7, AAV8, AAV9, AAV10, AAV11, AAV12, AAV13, or AAV-DJ.

In certain embodiments, the tropism of the AAV include skeletal muscle (such as AAV1, AAV6, AAV7, AAV8, or AAV9, preferably AAV9).

In certain embodiments, the AAV ITR, the AAV Rep, and the AAV Cap are from the same or different AAVs.

In certain embodiments, the AAV ITR is AAV2 ITR, the AAV Rep is Rep2 from AAV2, and the AAV Cap is Cap9 from AAV9 or a derivative thereof.

In certain embodiments, the coding sequence for AAV Rep and Cap proteins is under the transcriptional control of a promoter, such as an AAV p5 promoter, a modified p5 promoter lacking RBE (Rep-Binding Element), or a ubiquitous promoter (such as CMV promoter, EF1a promoter, CAG promoter, CB promoter etc).

In certain embodiments, the AAV helper genes comprise adenoviral, herpesviral, or papillomaviral genes useful for AAV packaging (such as E1A, E1B, E2A, E4 and VA RNA), optionally operably linked to a promoter as one transcriptional unit.

In certain embodiments, the helper virus gene comprises: (i) an adenovirus gene, optionally an Adenovirus 5 or Adenovirus 2 gene; and/or (ii) a VA nucleic acid encoding functional VA RNA I and II, an E2A gene encoding a functional E2A protein, and an E4 gene encoding a functional E4 protein.

Another aspect of the invention provides a composition or a kit, comprising the recombinant DNA vector of the invention. In some embodiments, the kit comprises a plasmid comprising the recombinant DNA vector of the invention.

Another aspect of the invention provides a host cell comprising the recombinant DNA vector of the invention, or the composition of the invention.

In certain embodiments, the host cell is a HEK293 cell (such as an Expi293F cell), a HeLa cell, an A549 cell, a BHK cell, a VP2 cells, or an insect cell (such as Sf9).

In another aspect, the invention provides a method of propagating, amplifying, or producing a recombinant replication-defective AAV viral particle encapsidating the GOI, comprising introducing the subject recombinant DNA vector (single transfection vector) (e.g., in a plasmid) into a host cell.

In certain embodiments, the method further comprises harvesting the recombinant replication-defective AAV viral particle from the host cell.

In certain embodiments, the host cell is a HEK293 cell (such as an Expi293F cell), a HeLa cell, an A549 cell, a BHK cell, VP2 cells, or an insect cell (such as Sf9).

In certain embodiments, the recombinant DNA vector is introduced to the host cell by transient transfection.

With the general principles of the invention set forth herein, the sections below provides further detailed description for various aspects of the invention. It should be understood that any embodiment of the invention can be combined with any one or more additional embodiments of the invention, including those embodiments described in different sections of the application, and those described only in the examples, drawings, or claims.

Furthermore, all disclosures pertaining to “recombinant DNA vector” are also applicable to a “vector” and a “plasmid” described herein.

### 2. Rep and Cap Genes

In certain embodiments, the coding sequence for the AAV Rep and Cap proteins, Helper genes and the gene of interest (GOI) flanked by AAV ITR sequences, are integrated into a single transfection vector, e.g., a single plasmid.

The Rep proteins are important for replication and packaging, while the capsid proteins are assembled to create the protein shell of the AAV, or AAV capsid, which form the outer capsid shell that protects the viral genome, as well as being actively involved in cell binding and internalization. Alternative splicing and alternate initiation codons and promoters result in the generation of four different Rep proteins (Rep78, Rep68, Rep52, and Rep40) from a single open reading frame and the generation of three capsid proteins (VP; VP1/VP2/VP3) from a single open reading frame. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype.

In certain embodiments, the AAV ITR, the AAV Rep, and the AAV Cap are from the same or different AAV serotypes.

In some embodiments, the AAV ITR, the AAV Rep, and the AAV Cap are from AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9 K449R, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP 63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In certain embodiments, the AAV Cap is a derivative of wild-type AAV9. In certain embodiments, the derivative comprises an insertion of a short peptide (e.g., 3, 4, 5, 6, 7, 8, or 9 residues) in-between residues 588 and 589 of the wild-type AAV 9 capsid VP1. In certain embodiments, the insertion comprises, consists essentially of, or consists of RGDLGLS into residues 588 and 589 of wild-type AAV9 Capsid VP1.

In certain embodiments, the rDNA vector/plasmid of the invention comprises a cap gene promoter. The cap gene promoter may be operably linked to a cap gene. In certain embodiments, the cap gene promoter is a native cap gene promoter.

The native cap gene (i.e., the cap gene of a wild type AAV) is operably linked to a p40 promoter, a p5 promoter and a p19 promoter. Thus in one embodiment, the rDNA vector/plasmid of the invention comprises a cap gene promoter such as an AAV p40 promoter, a p5 promoter, and/or a p19 promoter.

The native p40 promoter is contained within the native rep gene. In some embodiments, the p40 promoter has a sequence at least 95%, at least 98%, or 99% identical to that of AAV2. In some embodiments, the at least one cap gene promoter is comprised in a promoter region comprising a p40 promoter, a p5 promoter and a p19 promoter.

The native p5 promoter is upstream of the native rep gene. In some embodiments, the p5 promoter has a sequence at least 95%, at least 98%, or at least 99% identical to that of AAV2.

The p19 promoter is contained within the native rep gene. In some embodiments, the p19 promoter has a sequence at least 95%, at least 98%, or at least 99% identical to that of AAV2.

In some embodiments, the AAV ITR, the AAV Rep and the AAV Cap are from the same AAV. In some other embodiments, the AAV ITR, the AAV Rep and the AAV Cap are from different AAVs. For example, the AAV Rep may be from AAV2, and the AAV Cap may be from AAV9 or a derivative thereof such as the aforementioned wt AAV9 derivatives, and including SLB-101.

In certain embodiments, the AAV ITR is AAV2 ITR, the AAV Rep is Rep2 from AAV2, and the AAV Cap is Cap9 from AAV9 (or a derivative thereof).

In certain embodiments, the coding sequence for AAV Rep protein encodes a wild type Rep 40, Rep 52, Rep 68 and/or Rep 78 of an AAV, such as AAV2. In certain embodiments, these Rep proteins are transcribed from one or more of the rep promoters p5, p19 and/or p40.

The rep gene encodes at least one functional Rep proteins (Rep 78, Rep 68, Rep 52 and Rep 40). The gene region is under the control of the p5 and p19 promoters. When the p5 promoter is used, a sequence that encodes Rep 78 and Rep 68 is transcribed. Rep 78 and Rep 68 are two alternative splice variants (Rep 78 comprises an intron that is excised in Rep 68). Similarly, when the p19 promoter is used, a sequence that encodes Rep 52 and Rep 40 is transcribed. Rep 52 and Rep 40 are alternative splice variants (Rep 52 comprises an intron that is excised in Rep 40). The four Rep proteins are known to be involved in replication and packaging of the viral genome, and are thus useful in rAAV production.

In certain embodiments, it is not necessary for all four Rep proteins to be present. In certain embodiments, at least one encoded large Rep protein (Rep 78 or Rep 68) and one encoded small Rep protein (Rep 52 or Rep 40) are present. Rep 78 can be toxic to cells, and Rep 78 does not need to be present in order for AAV replication to take place. Thus, in some embodiments, Rep78 is absent. In some embodiments, the rep gene does not encode or transcribe Rep 78. In certain embodiments, the rep gene encodes/transcribes Rep 68.

In certain embodiments, the rDNA vector/plasmid of the invention comprises at least one rep gene encoding: (a) a functional Rep 52 protein; (b) a functional Rep 40 protein; and/or (c) a functional Rep 68 protein.

As used herein, a “functional” Rep protein is one which allows for production of AAV particles. In particular, Rep 78 or Rep 68 (the large Rep proteins) are believed to be involved in replication of the AAV genome, and Rep 52 and Rep 40 (the small Rep proteins) are believed to be involved in packaging of the AAV genome into a capsid. One of skill in the art can readily determine whether a given Rep protein is functional, such as by determining, e.g., whether the Rep protein supports AAV production using an AAV production assay as described above.

In some embodiments, the at least one rep gene encodes a “functional” Rep protein if the Rep protein supports rAAV production at a level at least 25%, at least 40%, at least 50%, at least 70%, at least 80%, at least 90% or at least 95% of the level supported by the wild type Rep protein.

In certain embodiments, the Rep protein is compatible with the ITR(s) surrounding the GOI. Some Rep proteins may only be able to package genomic material (such as an expression cassette) when it is flanked by ITR(s) of the same serotype as the Rep protein. Other Rep proteins are cross compatible, and can package genomic material that is flanked by ITR(s) of a different serotype.

In certain embodiments, the rDNA vector/plasmid of the invention comprises two or more rep genes that are separated on the vector/plasmid. For example, one of the separate rep genes could encode Rep 68 (for example using the p5 promoter or a different promoter situated near the normal position of the p5 promoter in the rep gene), another encoding Rep 40 (for example using the p19 promoter or a different promoter situated near the normal position of the p19 promoter). The one rep gene encoding Rep 40 may also encode Rep 52 since Rep 52 and Rep 40 are alternative splice variants.

In certain embodiments, if two genes encode a Rep 40 protein, one of the two genes that encodes a functional Rep 40 protein may comprise an intron. In one embodiment, both genes that encode a functional Rep 40 protein comprise an intron. In another embodiment, only one of the genes that encodes a functional Rep 40 protein comprises an intron.

For example, to avoid the at least one rep gene encoding Rep 78, the rep gene may be split through partial duplication into two genes. One gene could comprise nucleotides corresponding to the full length native rep gene with the sequence corresponding to the intron removed. Such a gene would encode Rep 68 and Rep 40, but would not encode either Rep 78 or Rep 52, as a portion of each of the Rep 78 and Rep 52 proteins is encoded by the sequence which acts as an intron in the context of rep 40. The second gene could comprise nucleotides corresponding to the region of the native rep gene downstream of the p19 promoter, which would encode Rep 52 (intron spliced in) and Rep 40 (intron spliced out).

In some embodiments, the at least one rep gene does not comprise a functional internal p40 promoter. The native rep/cap gene comprises a p40 promoter (Pereira and Muzyczka, J. Virol. 71:1747-1756, 1997). The p40 promoter drives expression of the cap gene, but is not required for expression of rep genes. A functional p40 promoter is one that is capable to drive expression of the cap gene.

In certain embodiments, the coding sequence for AAV Rep and Cap proteins is under the transcriptional control of a promoter, such as an AAV p5, p19, or p40 promoter, a modified p5 promoter lacking RBE (Rep-Binding Element), or a ubiquitous promoter (such as CMV promoter, EF1a promoter, CAG promoter, CB promoter etc).

In any of the above embodiments, either the rep gene or the cap gene or both may be replaced by a cloning site (e.g., a multicloning site or MCS), in the subject rDNA vector, such that a suitable rep and/or cap gene can be cloned/inserted into the respective cloning sites. This will facilitate the swapping of any desired rep or cap genes into the subject rDNA vector/plasmid.

### 3. Gene of Interest (GOI) in RAAV and Treatable Diseases

The system and method of the invention can be used to produce recombinant AAV vectors carrying a gene of interest (GOI) flanked by AAV ITR sequences.

In certain embodiments, the rDNA vector (plasmid) of the invention comprises ITR sequences derived from AAV1, AAV2, AAV4 and/or AAV6. In certain embodiments, the ITR sequences are AAV2 ITR sequences.

As used herein, “gene of interest” or GOI or goi generally refers to a nucleic acid or polynucleotide sequence, such as a gene, an open reading frame (ORF), or a coding sequence for protein or RNA, such as non-coding RNA that includes siRNA, miRNA, shRNA, antisense RNA or a precursor thereof. However, in certain circumstances or context, the term GOI also loosely refers to a protein (encoded by the GOI), or a disease or indication that can be remedied by the GOI, or a disease or indication can be (but is not necessarily) caused by loss of function of the GOI.

For example, and merely to illustrate, the gene GALGT2 encodes the protein GalNAc transferase (β-1,4-N-acetylgalactosamine galactosyltransferase), which is an enzyme that transfers a complex sugar molecule onto a few specific proteins, including dystroglycan.

Under normal circumstances, GalNAc transferase is found only at the neuromuscular junction (NMJ), where some components of the dystroglycan-associated protein complex are different than elsewhere in muscle. Importantly, at the NMJ, utrophin is present instead of dystrophin. In the mdx mouse model of muscular dystrophy, viral gene transfer of GALGT2 results in expression of GalNAc transferase across the entire muscle membrane, instead of just at the normal expression domain of the NMJ, as well as upregulation of utrophin across the entire muscle fiber. In the mdx mouse, this expression can correct muscle functional deficits to the same degree as does microdystrophin gene expression. Furthermore, overexpression of GALGT2 corrects muscle pathology in mouse models of other muscular dystrophies, including LGMD2A and congenital muscular dystrophy (MDC1A). Thus GALGT2 is a GOI for treating muscular dystrophy such as DMD, BMD, LGMD2A and MDC1A, even though GALGT2 is not necessarily defective per se in the patient in need of treatment.

In another example, Sarcolipin (SLN) inhibits the sarco/endoplasmic reticulum (SR) Ca²⁺ ATPase (SERCA), and is abnormally elevated in the muscle of DMD patients and animal models such as the mdx mouse model of DMD. Reducing SLN levels by AAV9-mediated RNA interference ameliorates dystrophic pathology in the severe dystrophin/utrophin double mutant (mdx:utr^{-/-}) mouse model of DMD, including attenuation of muscle pathology and improvement of diaphragm, skeletal muscle and cardiac function. Thus the coding sequence for SLN RNAi is a GOI that remedies DMD.

Thus the GOI can be a gene (or protein) that, when expressed, replaces a mutated, damaged, or inactive gene or protein. The GOI can be a gene (or protein) that, when expressed, assists an already functioning process that can benefit from further modification for therapy in a disease, disorder, or dysfunction. The GOI can be a gene (or protein) that, when expressed, assists a dysfunctional process that can benefit from further modification for therapy in a disease, disorder, or dysfunction. A GOI nucleic acid sequence can be DNA, RNA, or synthetic nucleic acid molecule. The GOI can be a protein, an enzyme, a structural protein, a functional protein, or an adaptable protein based on cell function(s). The GOI can provide therapeutic benefit or a treatment modality for a disease, disorder, or dysfunction.

In certain embodiments, the recombinant DNA vector of the invention comprises more than one copy of the GOI. In some embodiments, the plasmid of the invention comprises more than one copy of the GOI. In certain embodiments, the multiple GOI copies are the same. In some other embodiments, the multiple GOI are different.

In certain other embodiments, the GOI may be a CRISPR/Cas effector enzyme, such as a Class 2, Type II, IV, V, or VI effector enzyme, including CRISPR-Cas9, Cas 12, Cas 13, etc. In certain embodiments, the GOI may be a TALEN, or other genetic based gene editing protein that functions upon intracellular delivery for their intended activity, such as gene editing or gene knockout in a target cell, tissue, or organism/individual.

In certain embodiments, CRISPR/Cas effector enzyme lacks endonuclease activity (dCas, such as dCas9).

In certain embodiments, the Cas or dCas is further fused to a base editor, such as a cytosine base editor (CBE, e.g., APOBEC, BE1, BE2, BE3, Target-AID base editor, SaBE3, BE3 PAM variants, BE3 editing window variants, AID, CDA1, APOBEC3G, HF-BE3, BE4, BE4max, and AncBE4max), an adenine base editor (ABE, e.g., ABE7.10, ABE 6.3, ABE7.8, ABE7.9, ABEmax, ABE8e(TadA-8e V106W), ABE8 and variants thereof), or a dual base editor (SPACE, A&C-BEmax).

Any and all GOIs as used herein may benefit from codon optimization for enhanced expression and activity via known computer-based algorithms or other codon optimization method, such as manual optimization.

In certain embodiments, the GOI is within a pro-AAV cassette comprising the GOI operably linked to a promoter.

In some embodiments, the promoter operably linked to the GOI is an ubiquitous promoter which drives or promotes expression of the GOI in most tissues. In some embodiments, the promoter is elongation factor 1α-subunit (EF1α) promoter, cytomegalovirus (CMV) immediate-early enhancer promoter, chicken β-actin (CBA) and its derivative CAG promoter, β glucuronidase (GUSB) promoter, or ubiquitin C (UBC) promoter.

In some other embodiments, the promoter operably linked to the GOI is a tissue-specific promoter which can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

Non-limiting examples of muscle-specific promoters include mammalian muscle creatine kinase (MCK) promoter, mammalian desmin (DES) promoter, mammalian troponin I (TNNI2) promoter, and mammalian skeletal alpha-actin (ASKA) promoter.

In some embodiments, the pro-AAV cassette may further comprise an enhancer that promotes the transcription of the GOI from the promoter, a 5′ UTR, a Kozak sequence, a heterologous intron that promotes transcription and/or translation of the GOI, a 3′ untranslated region (UTR), a WPRE sequence, and/or a polyA signal sequence.

In certain embodiments, the pro-AAV cassette comprises a transcription regulatory element comprising the promoter element and/or enhancer element from HLP2, HLP1, LP1, HCR-hAAT, ApoE-hAAT, and/or LSP. These transcription regulatory elements are described in more detail in the following references: HLP2: WO16/075473; HLP1: McIntosh et al., Blood 121(17):3335-44, 2013; LP1: Nathwani et al., Blood 107(7):2653-2661, 2006; HCR-hAAT: Miao et al., Mol Ther. 1:522-532, 2000; ApoE-hAAT: Okuyama et al., Human Gene Therapy 7:637-645, 1996; and LSP: Wang et al., Proc Natl Acad Sci U S A. 96(7):3906-3910, 1999 (all incorporated herein by reference). Each of these transcription regulatory elements may comprise a promoter, an enhancer, and/or optionally other nucleotides.

In certain embodiments, the plasmid of the invention comprises a promoter region comprising one or more promoters, and the promoter region does not comprise dispensable translation initiation codons (e.g., ATG or GTG codons) to prevent unintended/undesirable translation initiation. In certain embodiments, the promoter region comprises p5, p19 and/or p40 promoters, and wherein ATG or GTG codons at one or more positions within these promoters are absent or mutated.

The GOI can be any gene or coding sequence within the packaging capacity of the AAV, e.g., about 4-5 kb, or about 4.7 kb including the ITR sequences, or about 4.4 kb without accounting for the ITR sequences.

The AAV ITR sequences flanking the GOI can be from any AAV ITR, and can be from the same or different AAV serotypes. In certain embodiments, the AAV ITR sequences flanking the GOI are both from AAV1, AAV2, AAV3A, AAV3B, AAV4, AAV5, AAV6, AAV7, AAV8, AAV9, AAV10, AAV11, AAV12, AAV13, or AAV-DJ.

In some embodiments, the AAV ITR, the AAV Rep and the AAV Cap are from the same AAV. In some other embodiments, the AAV ITR, the AAV Rep and the AAV Cap are from different AAVs.

In certain embodiments, the AAV ITR is AAV2 ITR, the AAV Rep is Rep2 from AAV2, and the AAV Cap is Cap9 from AAV9 (or a derivative thereof).

In certain embodiments, the tropism of the AAV include skeletal muscle (such as AAV1, AAV6, AAV7, AAV8, or AAV9, preferably AAV9, or a derivative thereof, such as SLB-101).

In certain aspects, the rAAV that may be produced by using the subject single vectors (e.g., single plasmids) and the suitable host cells (which can supply any useful helper functions for rAAV production in trans), may encode a gene of interest (GOI) useful for, e.g., gene therapy to treat a disease or condition.

Representative (non-limiting) gene of interest (GOI) may include: a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a microdystrophin gene.

In certain embodiments, the microdystrophin gene is any such one described in the following patents: U.S. Pat. Nos. 7,906,111; 7,001,761; 7,510,867; 6,869,777; 8,501,920;7,892,824; PCT/US2016/013733; U.S. Pat. No. 10,166,272 (all incorporated herein by reference). In certain embodiments, the microdystrophin gene is capable of being packaged into a rAAV virion, e.g., no more than about 4.7 kb in size.

In certain embodiments, the microdystrophin gene contains within its coding sequence spectrin-like repeats R16 and R17 that are capable of restoring nitric oxide synthase (nNOS) activity to the sarcolemma (such as those described in U.S. Pat. No. 7,892,824).

In certain embodiments, the microdystrophin gene comprises a coding sequence for the R1, R16, R17, R23, and R24 spectrin-like repeats (i.e., SR1, SR16, SR17, SR23, and SR24, respectively) of the full-length dystrophin protein, such as one described in PCT/US2016/013733 (incorporated herein by reference). In certain embodiments, the microdystrophin gene does not encode any other spectrin repeats of the full-length dystrophin protein, other than SR1, SR16, SR17, SR23, and SR24.

Diseases or conditions having a potential to benefit from the rAAV produced by the subject single transfection vector include: Huntington's disease, X-linked myotubular myopathy (XLMTM), Acid maltase deficiency (e.g., Pompe disease), Spinal Muscular Atrophy (SMA), Myasthenia Gravis (MG), Amyotrophic lateral sclerosis (ALS), Friedreich's ataxia, Mitochondrial myopathy, Muscular dystrophies (Duchenne's muscular dystrophy, Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), Mucopolysaccharidoses (MPS), Metachromatic leukodystrophy (MLD), Batten Disease, Rett Syndrome, Krabbe Disease, Canavan disease, X-Linked Retinoschisis, Achromatopsia (CNGB3 and CNGA3), X-Linked Retinitis Pigmentosa, Age-Related Macular Degeneration, neovascularized macular degeneration, Pompe, Fabry's disease, MPS I, II, IIIA, IIIB, Gaucher's disease, Dannon Disease, A1At Deficiency, Friedreich ataxia, Wilson's Disease, Batten Disease (CLN1, CLN3, CLN6, CLN8), Wolman Disease, Tay-Sachs, Niemann-Lick Type C, CDKL5 deficiency Disorder, B-thalassemia, Sickle cell disease.

In certain embodiments, diseases or conditions having a potential to benefit from the rAAV produced by the subject single transfection vector may include: Becker muscular dystrophy (BMD), Congenital muscular dystrophies (CMD), Bethlem CMD, Fukuyama CMD, Muscle-eye-brain diseases (MEBs), Rigid spine syndromes, Ullrich CMD, Walker-Warburg syndromes (WWS), Duchenne muscular dystrophy (DMD), Emery-Dreifuss muscular dystrophy (EDMD), Facioscapulohumeral muscular dystrophy (FSHD), Limb-girdle muscular dystrophies (LGMD), Myotonic dystrophy (DM), Oculopharyngeal muscular dystrophy (OPMD), Motor neuron diseases including ALS (amyotrophic lateral sclerosis), Spinal-bulbar muscular atrophy (SBMA), Spinal muscular atrophy (SMA).

In certain embodiments, diseases or conditions having a potential to benefit from the rAAV produced by the subject single transfection vector may include ion channel diseases, which are typically marked by muscular weakness, absent muscle tone, or episodic muscle paralysis. They include Andersen-Tawil syndrome, Hyperkalemic periodic paralysis, Hypokalemic periodic paralysis, Myotonia congenita, Becker myotonia, Thomsen myotonia, Paramyotonia congenita, Potassium-aggravated myotonia.

In certain embodiments, diseases or conditions having a potential to benefit from the rAAV produced by the subject single transfection vector may include mitochondrial diseases, which occur when structures that produce energy for a cell malfunction. Such diseases include: Friedreich's ataxia (FA), Mitochondrial myopathies, Kearns-Sayre syndrome (KSS), Leigh syndrome (subacute necrotizing encephalomyopathy), Mitochondrial DNA depletion syndromes, Mitochondrial encephalomyopathy, lactic acidosis and stroke-like episodes (MELAS), Mitochondrial neurogastrointestinal encephalomyopathy (MNGIE), Myoclonus epilepsy with ragged red fibers (MERRF), Neuropathy, ataxia and retinitis pigmentosa (NARP), Pearson syndrome, Progressive external opthalmoplegia (PEO).

In certain embodiments, diseases or conditions having a potential to benefit from the rAAV produced by the subject single transfection vector may include myopathies, which is a disease of muscle in which the muscle fibers do not function properly, resulting in muscular weakness. Myopathies include: Cap myopathies, Centronuclear myopathies, Congenital myopathies with fiber type disproportion, Core myopathies, Central core disease, Multiminicore myopathies, Myosin storage myopathies, Myotubular myopathy, Nemaline myopathies, Distal myopathies, GNE myopathy/Nonaka myopathy/hereditary inclusion-body myopathy (HIBM), Laing distal myopathy, Markesberg-Griggs late-onset distal myopathy, Miyoshi myopathy, Udd myopathy/tibial muscular dystrophy, Vocal cord and pharyngeal distal myopathy, Welander distal myopathy, Endocrine myopathies, Hyperthyroid myopathy, Hypothyroid myopathy, Inflammatory myopathies, Dermatomyositis, Inclusion-body myositis, Polymyositis, Metabolic myopathies, Acid maltase deficiency (AMD, Pompe disease), Carnitine deficiency, Carnitine palmityl transferase deficiency, Debrancher enzyme deficiency (Cori disease, Forbes disease), Lactate dehydrogenase deficiency, Myoadenylate deaminase deficiency, Phosphofructokinase deficiency (Tarui disease), Phosphoglycerate kinase deficiency, Phosphoglycerate mutase deficiency, Phosphorylase deficiency (McArdle disease), Myofibrillar myopathies (MFM), Scapuloperoneal myopathy.

In certain embodiments, diseases or conditions having a potential to benefit from the rAAV produced by the subject single transfection vector may include neuromuscular junction diseases, which result from the destruction, malfunction or absence of one or more key proteins involved in the transmission of signals between muscles and nerves. Such diseases include: Congenital myasthenic syndromes (CMS), Lambert-Eaton myasthenic syndrome (LEMS), Myasthenia gravis (MG).

In certain embodiments, diseases or conditions having a potential to benefit from the rAAV produced by the subject single transfection vector may include peripheral nerve diseases, in which the motor and sensory nerves that connect the brain and spinal cord to the rest of the body are affected, causing impaired sensations, movement or other functions. Such diseases include: Charcot-Marie-Tooth disease (CMT), Giant axonal neuropathy (GAN), muscle wasting in cachexia and aging.

In certain embodiments, the GOI coding sequence comprises a polyA signal sequence or polyadenylation site.

In certain embodiments, the polyadenylation site is a bovine growth hormone (bGH) polyadenylation site.

In certain embodiments, the polyadenylation site or poly(A) signal sequence is from other suitable sources, e.g., synthetic sequences or sequences from other eukaryotic genes or viruses.

In certain embodiments, the GOI coding sequence is partially or fully codon-optimized for expression in a mammalian host cell. For example, the most 3′ 300-350 nucleotides of the coding sequence may be codon-optimized for expression in the mammalian host cell.

### 4. Composition or Kits of Single Vector (e.g., Single Plasmid) System

One aspect of the invention provides a composition or a kit, comprising a single (e.g., single-plasmid) system that comprises, consists essentially of, or consists of any one of the single recombinant DNA vector of the invention.

The single vector (single plasmid) system in the subject composition or kit is useful for producing rAAV. The single vector (single plasmid) system in the subject composition or kit is suitable for use in producing rAAV. The single vector (single plasmid) system in the subject composition or kit is for producing rAAV. The single vector (single plasmid) system in the subject composition or kit is for producing rAAV suitable for use in gene therapy. The single vector (single plasmid) system in the subject composition or kit is for producing rAAV for use in gene therapy.

The phrase “single vector (single plasmid) system” refers to a system that comprises a vector (e.g., a single plasmid), and can be used together, without the need for additional plasmids to produce rAAV. In certain embodiments, the single vector (single plasmid) system can be used to produce rAAV without the need for helper virus such as adenovirus. In certain embodiments, the single vector (single plasmid) system can be used to produce rAAV without the need for genetic material originating from a host cell, optionally with the exception of a gene encoding El A/B. However, the vector system in the composition/kit of the invention may comprise additional non-plasmid components.

In certain embodiments, the single vector (single plasmid) system of the invention comprises all the necessary genetic information for the production of rAAV. For example, the single vector (single plasmid) system of the invention may comprise at least one rep gene, at least one cap gene and at least one helper gene. In certain embodiments, the single vector (single plasmid) system of the invention comprises all the necessary genetic information useful for the production of rAAV suitable for use in gene therapy. For example, the single vector (single plasmid) system of the invention may comprise at least one rep gene, at least one cap gene, at least one helper gene and an expression cassette comprising a transgene operably linked to at least one regulatory control element.

In certain embodiments, the helper vector may comprise at least one helper virus gene. AAV is only able to propagate in the presence of a helper virus. Examples of helper viruses include adenoviruses, and herpes viruses.

In certain embodiments, the helper vector of the invention comprise sufficient helper genes to allow for AAV replication and packaging. In certain embodiments, the helper vector that can be used with the rDNA vector of the invention provides all the helper genes/functions for AAV packaging (except for the ITR sequences flanking the GOI).

For example, for host cells expressing the adenoviral E1A/B genes, such as the HEK293T cells, the remaining adenoviral helper genes encode E4, E2A and VA RNA I and II. These helper genes can be included in the helper vector in any order or orientation, so long as all these genes can be transcribed and expressed to facilitate AAV packaging.

Whether a helper vector comprises sufficient helper genes to facilitate AAV production can be assessed using any art recognized AAV production assay to show sufficient titer of AAV so produced.

In one embodiment, the helper gene products will be considered (sufficient) to facilitate AAV production if they support rAAV production at a level at least 25%, at least 40%, at least 50%, at least 60%, at least 70%, at least 80%, at least 90% or at least 95% of the level supported by adenoviral helper genes encoding E4, E2A and VA RNA I and II. In certain embodiments, if the yield of rAAV produced is at least 25%, at least 40%, at least 50%, at least 60%, at least 70%, at least 80%, at least 90% or at least 95% of the yield of rAAV produced using the reference single plasmid system in which one is a helper vector comprising adenoviral helper genes encoding E4, E2A and VA RNA I and II.

Since the helper vector encode the genes that allow for efficient AAV production, the addition of a helper virus is not required (i.e., the composition or kit of the invention comprising the single vector/plasmid of the invention is sufficient to support AAV production and packaging).

In certain embodiments, the at least one helper virus gene comprises an adenovirus gene. Adenovirus is a virus which is known to aid propagation of AAV (Xiao et al., J. Virol, 72:2224-2232, 1998). In certain embodiments, the at least one helper virus gene comprises an Adenovirus 5 gene or an Adenovirus 2 gene. In certain embodiments, the helper genes of adenoviruses encode ElA, E1B, E4, E2A and VA RNA I and II.

ElA is encoded by nucleotides 560-1545 of the Adenovirus 5 genome. The E1A may optionally lack the non-essential intron at nucleotides 1113-1228.

E1B is actually two proteins E1B 19K and E1B 55K, which work together to block apoptosis in adenovirus-infected cells. E1B is encoded by nucleotides 1714-2244 (E1B 19K), and by nucleotides 2019-3509 (E1B 55 K) of the Adenovirus 5 genome.

E4 is encoded by a number of different open reading frames (ORFs) of the Adenovirus 5 genome. E4 ORF 6/7 is encoded by nucleotides 32914-34077, which may optionally lack the intron between nucleotides 33193 and 33903. E4 34K is encoded by nucleotides 33193-34077 of the Adenovirus 5 genome. E4 ORF 4 is encoded by nucleotides 33998-34342 of the Adenovirus 5 genome. E4 ORF 3 is encoded by nucleotides 34353-34703 of the Adenovirus 5 genome. E4 ORF B is encoded by nucleotides 34700 to 35092 of the Adenovirus 5 genome. E4 ORF 1 is encoded by nucleotides 35140-35526 of the Adenovirus 5 genome. A functional E4 protein may only comprise amino acids encoded by ORFs 6 and 7, as only the amino acids encoded by ORFs 6 and 7 are mandatory for activity. In certain embodiments, the functional E4 protein comprises a polypeptide sequence encoded by all or a significant portion of ORFs 6 and 7. Optionally, the functional E4 protein does not comprise polypeptide sequence encoded by all or a portion of ORFs 1-4 and 34K.

In certain embodiments, the functional E4 protein comprises amino acids encoded by ORFs 1-7, as the amino acids encoded by ORFs 1-3 and 34K of E 4, though not required, do improve the activity of the E4 protein.

The E2 (E2A) gene is encoded by nucleotides 22443-24032 of the Adenovirus 5 genome.

The VA RNA I and II is encoded by nucleotides 10589-11044 of the Adenovirus 5 genome.

E1B and E4 are believed to enhance AAV mRNA accumulation, and E2A and VA RNA I and II are believed to enhance AAV mRNA splicing and translation. E1B, E4 and E2A are proteins encoded by genes present in the adenovirus genome, whereas the VA nucleic acid encodes two RNA transcripts known as VA RNA I and VA RNA II. The transcripts themselves are functional in the cell, and are never translated into amino acid sequences. It will be appreciated, therefore, that the VA nucleic acid does not encode a protein, but does “encode” or “correspond to” an RNA, i.e., whilst the term “encode” is used the VA nucleic acid is a non-translated nucleic acid sequence.

Of the five adenovirus genes, in some embodiment, it is possible not to include ElA or E1B in the helper vector, as some host cell lines (such as HEK293 cells) express one or more of E1A or E1B constitutively. In certain embodiments, the helper vector does not comprise a gene encoding a functional adenoviral E1A/B protein.

In one embodiment, the at least one helper virus gene comprises: (a) a VA (viral associated) nucleic acid encoding functional VA RNA I and II; (b) an E2A gene encoding a functional E2A protein; and/or (c) a E4 gene encoding a functional E4 protein.

In certain embodiments, the at least one helper virus gene comprises a VA nucleic acid (such as VA RNA I and/or II), an E2A gene and an E4 gene.

A “functional” VA RNA I and II, E2A protein or E4 protein is able to facilitate production of AAV.

One of skill in the art can determine whether a given VA RNA I and II, E2A protein or E4 protein is functional by determining, for example, whether the VA RNA I and II, E2A protein or E4 protein supports AAV production using an AAV production assay as described above. In some embodiments, the VA RNA I and II, E2A protein or E4 protein is considered to be functional if it supports rAAV production at a level at least 25%, at least 40%, at least 50%, at least 70%, at least 80%, at least 90% or at least 95% of the level supported by the wild type (for example as found in native Adenovirus 5) VA RNA I and II, E2A protein or E4 protein. In certain embodiments, the E4 protein will be considered to be “functional” if it supports rAAV production at a level at least 70%, at least 80%, at least 90% or at least 95% of the level supported by the wild type E4 protein.

In certain embodiments, the E4 gene is not located between the VA nucleic acid and the E2A gene, i.e., the sequence of the plasmid is such that E4 gene sequence does not appear in the plasmid between the VA nucleic acid sequence and the E2A gene sequence. In certain other embodiments, the E2A gene is located between the VA nucleic acid and the E4 gene.

Any of the genes on the helper vector can take any orientation without regard to the other genes.

In certain embodiments, the VA nucleic acid has an activity level which has at least 75%, at least 80%, at least 90%, at least 95%, or between 95% and 100% of the activity of a wild type VA nucleic acid from Adenovirus 5. The activity level of the VA nucleic acid can be determined by, for example, measuring rAAV yield, for example using the AAV production assay described above.

Expression of the E4 gene is driven by a promoter (designated “E4 promoter”). A fully active promoter comprises nucleotides corresponding to 35793-35848 of the Adenovirus 5 genome. In certain embodiments, the E4 gene is operably linked to an E4 promoter that has at least 50%, at least 70%, or at least 90% of the activity of a wild type promoter from Adenovirus 5. The activity of the E4 promoter may be determined by testing its ability to drive expression of a protein. In one embodiment, the E4 promoter has at least 25%, at least 40%, at least 50%, at least 70%, or at least 90% of the activity of a wild type E4 promoter from Adenovirus 5 if it supports rAAV production at a level at least 25%, at least 40%, at least 50%, at least 60%, at least 70%, or at least 90% of the level supported by a wild type E4 gene promoter, i.e., if the yield of rAAV produced is at least 50%, at least 70%, or at least 90% of the yield of rAAV produced using the reference single-plasmid system comprising wild type helper genes with wild-type promoters. Preferably, the E4 promoter will be considered to facilitate AAV production if it supports rAAV production at a level at least 70%, at least 80%, at least 90% or at least 95% of the yield of rAAV produced using the reference single-plasmid system comprising wild type helper genes with wild-type promoters.

### 5. Host Cells

One aspect of the invention comprises a host cell comprising the single recombinant DNA vector (single transfection vector) of the invention.

In general, a host cell of the invention is capable or suitable for the production of rAAV. The host cell is typically derived from a eukaryotic cell line, such as a vertebrate cell line, including a mammalian cell line (e.g., a human cell line).

In certain embodiments, the host cell is a cell selected from the group consisting of a HEK293T cell, a HEK293 cell, a HEK293EBNA cell, a CAP cell, a CAP-T cell, an AGE1.CR cell, a PerC6 cell, a C139 cell, an EB66 cell, a BHK cell, a COS cell, a Vero cell, a Hela cell, and an A549 cell.

In certain embodiments, the host cell is selected from the group consisting of a HEK293T cell, a HEK293 cell, a HEK293EBNA cell, a CAP cell, a CAP-T cell, an AGE1.CR cell, a PerC6 cell, a C139 cell, and an EB66 cell.

In certain embodiments, the host cell is selected from the group consisting of a HEK293T cell, a HEK293 cell, and a HEK293EBNA cell.

In certain embodiments, the host cell is a HEK293T cell.

In certain embodiments, the host cell is a cell that expresses a functional adenoviral E1A/B protein. For example, the host cell may comprise a chromosome comprising a gene encoding a functional adenoviral El A/B protein.

The host cell is considered suitable or capable of producing recombinant AAV if it supports AAV production at a level at least 30%, at least 40%, at least 50%, at least 70%, at least 80%, at least 90% or at least 95% of the level supported by HEK 293T cells. In this case, the test single-vector/plasmid system of the invention can be introduced/transfected into the host cell whose suitability for the production of recombinant AAV is to be tested, and the reference single-vector/plasmid system is introduced/transfected into HEK293T cells.

In certain embodiments, the single transfection vector is stably integrated into the host cell genome.

In certain embodiments, the host cell is derived from a vertebrate, such as human, monkey, bovine, porcine, equine and other equids, canine, feline, ovine, goat, murine, rat, rabbit, mink, opossum, camel and other cameloids, chicken and other avian, armadillo, frog, or reptile, or derived from an insect cell.

In certain embodiments, the host cell is a cell line suitable for AAV packaging, such as Expi cells, HEK293 cells (or a derivative thereof such as HEK293T cells).

In certain embodiments, the host cell is a HEK293 cell (such as an Expi293F cell), a HeLa cell, an A549 cell, a BHK cell, or an insect cell (such as Sf9).

In certain embodiments, the host cell is HEK293 (human embryonic kidney), which can be grown using standard tissue culture media such as DMEM complemented with L-Gln, 5-10% fetal bovine serum (FBS), and 1% penicillin-streptomycin.

In some embodiments, the HEK293 cells are grown on a solid support, including tissue culture plates, dishes, flasks, and bottles. For growing adherent HEK293 cells, the percentage of FBS can be reduced during rAAV production in order to limit contamination by animal-derived components.

In some embodiments, the HEK293 cells are adapted to grow in suspension.

In certain embodiments, the host cell is a Vero cell, such as a Vero75.4 or V75 cell described herein. Such cells may grow on a solid support, including tissue culture plates, dishes, flasks, bottles, and microcarrier that allows the adherent Vero cells to grow in suspension-like conditions.

In certain embodiments, the host cell is a BHK (baby hamster kidney) cell, such as BHK21 or sBHK27. In certain embodiments, the BHK cells are adapted to grow in serum-free suspension.

In certain embodiments, the host cell is a HEK293 cell. In certain embodiments, the HEK293 cell is adapted for growth in serum-free media (such as F17 or Expi293 media) and in suspension, thus is amenable for large scale growth in a bioreactor. See, for example, Grieger et al. (Mol. Ther. 24:287-297, 2016, incorporated herein by reference).

In certain embodiments, the HEK293 cell is a HEK293T cell which expresses SV40 T antigen (the temperature sensitive allele tsA1609) and the neomycin/geneticin-resistance gene.

In certain embodiments, for production of rAAV particles, the host cell comprises helper virus proteins useful (e.g., required) for AAV packaging.

In some embodiments, the coding sequences of the helper virus proteins are introduced into the host cell in a plasmid through transfection. In some other embodiments, the coding sequences of the helper virus proteins are integrated into the host cell genome.

In certain embodiments, the host cell of the invention may be adapted for use in producing recombinant AAV vectors encoding a gene of interest (GOI), which may be used in gene therapy. See the section entitled “Recombinant AAV Production” below. In such embodiments, one or more rAAV production cell lines may be infected by the subject single vectors, such as vectors or plasmids encoding AAV Rep, Cap and Helper proteins.

In certain embodiments, such producer/host cell line for rAAV production is a HeLa- or A549-derived cell line transfected with the single vector/plasmid of the invention, optionally containing a drug selection marker.

In certain embodiments, such producer cell line for rAAV production is a Vero cell.

In certain embodiments, such producer cell line for rAAV production is a BHK cell.

In certain embodiments, such producer cell line for rAAV production is a HEK293 cell.

In certain embodiments, such producer/host cell line for rAAV production comprises a single vector/plasmid of the invention comprising the GOI flanked by the AAV ITR sequences. The GOI can be any one of the GOI described herein useful for gene therapy, such as a dystrophin minigene or a microdystrophin gene described in U.S. Pat. No. 7,906,111; 7,001,761; 7,510,867; 6,869,777; 8,501,920; 7,892,824; WO2020/086844; or U.S. Pat. No. 10,166,272, or in PCT/US2016/013733 (all incorporated herein by reference).

For example, PCT/US2016/013733 (WO2016/115543A2) provides a micro-dystrophin gene operatively connected to a regulatory cassette, wherein the micro-dystrophin gene encodes a protein comprising: an amino-terminal actin-binding domain; a β-dystroglycan binding domain; and a spectrin-like repeat domain, comprising at least four spectrin-like repeats, wherein two of the at least four spectrin-like repeats comprise a neuronal nitric oxide synthase binding domain. In certain embodiments, the at least four spectrin-like repeats include spectrin-like repeat 1 (SR1), spectrin-like repeat 16 (SR16), spectrin-like repeat 17 (SR17), and spectrin-like repeat 24 (SR24). In certain embodiments, the protein encoded by the micro-dystrophin gene further comprises at least a portion of a hinge domain, such as at least one of a Hinge 1 domain, a Hinge 2 domain, a Hinge 3 domain, a Hinge 4 domain, and a hinge-like domain. In certain embodiments, the micro-dystrophin gene comprises, in N- to C-terminal order: a Hinge 1 domain (H1); a spectrin-like repeat 1 (SR1); a spectrin-like repeat 16 (SR16); a spectrin-like repeat 17 (SR17); a spectrin-like repeat 24 (SR24); and a Hinge 4 domain (H4). In certain embodiments, H1 is directly coupled to the SR1. In certain embodiments, SR 1 is directly coupled to SR16. In certain embodiments, SR16 is directly coupled to SR17. In certain embodiments, SR 17 is directly coupled to SR24. In certain embodiments, SR24 is directly coupled to the H4. In certain embodiments, the protein encoded by the micro-dystrophin gene further comprises between SR1 and SR16, in N- to C-terminal order, a spectrin-like repeat 2 (SR2) and a spectrin-like repeat 3 (SR3). In certain embodiments, SR1 is directly coupled to SR2 and SR2 is further coupled to SR3. In certain embodiments, H1 is directly coupled to SR1, SR1 is directly coupled to SR16, SR16 is directly coupled to SR17, SR17 is directly coupled to SR23, SR23 is directly coupled to SR24, and SR24 is directly coupled to H4.

In certain embodiments, the regulatory cassette is selected from the group consisting of a CK8 promoter and a cardiac troponin T (cTnT) promoter. In certain embodiments, the protein encoded by the micro-dystrophin gene has between five spectrin-like repeats and eight spectrin-like repeats. In certain embodiments, the protein encoded by the micro-dystrophin gene has at least 80% or 90% sequence identity to the amino acid sequence of SEQ ID NO: 4 or 5 in WO 2016/115543A2 (incorporated herein by reference).

### 6. Recombinant AAV Production

The recombinant DNA vector (single transfection vector) of the present invention, as well as the production cell lines, can be used for large scale production of recombinant AAV vectors (rAAV) useful for gene therapy. In certain embodiments, the system and the associated method of use, can be used for propagating/amplifying/producing of AAV viral particles comprising the ITR-flanked GOI.

Recombinant replication-defective AAV vectors, which can be produced with the present recombinant DNA vectors (single transfection vectors) and production cell lines, typically comprise a gene of interest (GOI) and expression regulators (such as promoters for the GOI) in lieu of the wild-type AAV virus rep and cap open reading frames (ORFs). The AAV rep and cap ORFs, optionally their native promoters p5, p19, and p40, and other helper functions useful for AAV packaging are supplied by the subject single transfection vector.

The rep ORF encodes four nonstructural Rep proteins involved in the AAV viral life cycle, and the cap ORF encodes the three structural proteins (i.e., VP1, VP2, and VP3) that form the icosahedral AAV capsid. Typically, the only AAV viral sequences that are retained in the rAAV vector genome are the inverted terminal repeats (ITRs)—the minimal cis-acting elements for AAV DNA replication and packaging.

In certain aspects, the method of propagating/amplifying/producing a replication-defective AAV particle encapsidating the GOI comprises introducing the subject recombinant DNA into a host cell, prior to, concurrently with, or subsequent to introducing the AAV helper genes for virus packaging.

In some embodiments, the recombinant DNA vector is introduced to the host cell by transient transfection.

The method for producing a replication-defective AAV particle comprises the steps of transfecting mammalian cells, such as, but not limited to HEK293 cells, with the subject recombinant DNA.

In certain embodiments, the host cell is derived from a vertebrate, such as human, monkey, bovine, porcine, equine and other equids, canine, feline, ovine, goat, murine, rat, rabbit, mink, opossum, camel and other cameloids, chicken and other avian, armadillo, frog, or reptile, or derived from an insect cell. Human cells include BHK cells, Vero cells, HEK293 cells, etc.

In certain embodiments, the host cell is a HEK293 cell (such as an Expi293F cell), a HeLa cell, an A549 cell, a BHK cell, or an insect cell (such as Sf9).

In certain embodiments, the host cell is HEK293 (human embryonic kidney), which can be grown using standard tissue culture media such as DMEM complemented with L-Gln, 5-10% fetal bovine serum (FBS), and 1% penicillin-streptomycin.

In some embodiments, the HEK293 cells are grown on a solid support, including tissue culture plates, dishes, flasks, and bottles. For growing adherent HEK293 cells, the percentage of FBS can be reduced during rAAV production in order to limit contamination by animal-derived components.

In some embodiments, the HEK293 cells are adapted to grow in suspension.

In certain embodiments, the HEK293 cell is adapted for growth in serum-free media (such as F17 or Expi293 media) and in suspension, thus is amenable for large scale growth in a bioreactor. See, for example, Grieger et al. (Mol. Ther. 24:287-297, 2016, incorporated herein by reference).

In certain embodiments, the HEK293 cell is a HEK293T cell which expresses SV40 T antigen (the temperature sensitive allele tsA1609) and the neomycin/geneticin-resistance gene.

In certain embodiments, the host cell is a Vero cell, such as a Vero75.4 or V75 cell described herein. Such cells may grow on a solid support, including tissue culture plates, dishes, flasks, bottles, and microcarrier that allows the adherent Vero cells to grow in suspension-like conditions.

In certain embodiments, the host cell is a BHK (baby hamster kidney) cell, such as BHK21 or sBHK27. In certain embodiments, the BHK cells are adapted to grow in serum-free suspension.

In some embodiments, the method further comprises harvesting the replication-defective AAV particle comprising the GOI. In some embodiments, the method comprises purifying the recombinant replication-defective AAV particle.

The gene of interest (GOI) may include genes useful for gene therapy in treating certain diseases or conditions. Representative (non-limiting) GOI may include a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

Suitable microdystrophin genes include those described in the following patents: U.S. Pat. Nos. 7,906,111; 7,001,761; 7,510,867; 6,869,777; 8,501,920; 7,892,824; PCT/US2016/013733; U.S. Pat. No. 10,166,272; WO2020/086844 (all incorporated herein by reference).

Diseases or conditions having a potential to benefit from the rAAV produced by the subject single vector-based system include: Huntington's disease, X-linked myotubular myopathy (XLMTM), Acid maltase deficiency (e.g., Pompe disease), Spinal Muscular Atrophy (SMA), Myasthenia Gravis (MG), Amyotrophic lateral sclerosis (ALS), Friedreich's ataxia, Mitochondrial myopathy, Muscular dystrophies (Duchenne's muscular dystrophy, Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), Mucopolysaccharidoses (MPS), Metachromatic leukodystrophy (MLD), Batten Disease, Rett Syndrome, Krabbe Disease, Canavan disease, X-Linked Retinoschisis, Achromatopsia (CNGB3 and CNGA3), X-Linked Retinitis Pigmentosa, Age-Related Macular Degeneration, neovascularized macular degeneration, Pompe, Fabry's disease, MPS I, II, IIIA, IIIB, Gaucher's disease, Dannon Disease, AlAt Deficiency, Friedreich ataxia, Wilson's Disease, Batten Disease (CLN1, CLN3, CLN6, CLN8), Wolman Disease, Tay-Sachs, Niemann-Lick Type C, CDKL5 deficiency Disorder, B-thalassemia, Sickle cell disease, etc.

Being a naturally replication-defective human parvovirus, wild-type AAV integrates its genome site-specifically within the host cell chromosome in the absence of helper assistance for its replication, where it persists indefinitely unless rescued via cellular infection with a helper virus. The introduction of a helper virus into the host cell triggers AAV replication and the generation of progeny virions. In the case of rAAV virions useful for gene therapy, introduction of the helper virus function into a suitable host cell triggers the packaging of the GOI in the rAAV virions, when the requisite rep and cap coding sequences are also supplied in the same system.

In other words, production of recombinant AAV relies on (1) the presence of the AAV rep and cap coding sequences, and (2) the helper virus functions.

In certain embodiments, the tropism of the AAV include serotypes such as AAV1, AAV2, AAV6, AAV7, AAV8, or AAV9, AAV10, AAV11, preferably AAV9. In certain embodiments, AAV capsids may be genetically modified, or capsids may be synthetic, designer capsids that enhance tissue specific or physiologic compartments delivery of a GOI to a specific tissue such as muscle, skeletal muscle, cardiac muscle, smooth muscle, and the like, as described (see, e.g., Zinn and Grimm, High-Throughput Dissection of AAV-Host Interactions: The Fast and the Curious, JMB 430(17):2626-2640, 2018; Kotterman and Schaffer, Engineering adeno-associated viruses for clinical gene therapy. Nature Reviews Genetics (2014) 4445-4451, both incorporated herein by reference). Tropism of AAV through pseudotyping, or the mixing of a capsid and genome from different viral serotypes may also be employed. These serotypes are denoted using a slash, so that AAV2/5 indicates a virus containing the genome of serotype 2 packaged in the capsid from serotype 5. Use of these pseudotyped viruses can improve transduction efficiency, as well as alter tropism. For example, pseudotyped AAV2/5 targets myoblasts (Duan et al., Enhancement of muscle gene delivery with pseudotyped adeno-associated virus type 5 correlated with myoblast differentiation. J Virol 75(16):7662-7671, 2001). Other pseudotyped AAV includes AAV2/6. In certain embodiments, In-silico-derived sequences were synthesized de novo and characterized for biological properties relevant to clinical applications. This effort led to the generation of nine functional putative ancestral AAVs and the identification of Anc80, the predicted ancestor of the widely studied AAV serotypes 1, 2, 8, and 9, as a highly potent in vivo gene therapy vector for targeting liver, muscle, and retina (Zinn et al., In Silico Reconstruction of the Viral Evolutionary Lineage Yields a Potent Gene Therapy Vector, Cell Reports 12(6):1056-1068, 2015); Buning et al., Engineering the AAV capsid to optimize vector-host-interactions, Current Opinion in Pharmacology, 24:94-104, 2015).

In certain embodiments, the tropism of the AAV include skeletal muscle (such as AAV1, AAV6, AAV7, AAV8, or AAV9, preferably AAV9).

In certain embodiments, the gene of interest (GOI) includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3 (CAPN-3), acid alpha-glucosidase (GAA, for Pompe disease), alpha-galactosidase A or GLA (for Fabry disease), glucocerebrosidase, dystrophin or microdystrophin.

In certain embodiments, the GOI is a functional equivalent of dystrophin (e.g., a dystrophin minigene encoding a functional micro-dystrophin protein).

In certain embodiments, the GOI is a microdystrophin gene.

In certain embodiments, the microdystrophin gene is one described in U.S. Pat. Nos. 7,906,111; 7,001,761; 7,510,867; 6,869,777; 8,501,920; 7,892,824; PCT/US2016/013733; or U.S. Pat. No. 10,166,272.

In certain embodiments, the microdystrophin gene comprises a coding sequence for R16 and R17 spectrin-like repeats for the full length dystrophin protein (such as one described in U.S. Pat. No. 7,892,824).

In certain embodiments, the microdystrophin gene comprises a coding sequence for the R1, R16, R17, R23, and R24 spectrin-like repeats of the full-length dystrophin protein (such as the microdystrophin gene described in PCT/US2016/013733).

In certain embodiments, the microdystrophin gene does not comprise coding sequence for and spectrin repeats of the full-length dystrophin protein other than the SR1, SR16, SR17, SR23, and SR24 repeats (e.g., in that order).

In certain embodiments, the subject rAAV vectors are produced in in vitro culture conditions, such as in bioreactors (e.g., 0.5L, 1L, 2L, 3L, 5L, 10L, 20L, 50L, 100L, 250L, 500L, or 1,000L working volume bioreactors), such as a CelliGen Plus packed-bed bioreactor (New Brunswick Scientific) for fed-batch vector production for 3 days post infection.

In certain embodiments, the recombinant replication-defective AAV viral particles are produced as in vitro culture on adherence-dependent cell lines, such as Vero and Vero-derived cell lines or HEK293 cells and HEK293-derived cells, that rely on a solid support. In certain embodiments, the solid support is a tissue culture surface, such as tissue culture dishes, plates, bottles, flasks, cell factory, etc. In certain embodiments, the solid support is a microcarrier, such as Cytodex 1 (GE Healthcare Life Sciences, Piscataway, NJ); a macrocarrier, such as FibraCel (New Brunswick Scientific, Edison, NJ), or a multilayered culture vessel, such as a CellCube (Corning Life Sciences, Lowell, MA) that permit medium perfusion.

In certain embodiments, the recombinant replication-defective AAV viral particles are produced as in vitro culture in eukaryote cells adapted to grow in suspension, such as a suspension culture of the BHK cell line or HEK293 cell line adapted for growth in suspension.

In certain embodiments, the culture supernatant yields in excess of 1×10¹⁰ plaque-forming units (PFU) of rAAV, 1×10¹¹ plaque-forming units (PFU) of rAAV, 1×10¹² plaque-forming units (PFU) of rAAV, 1×10¹³ plaque-forming units (PFU) of rAAV, 1×10¹⁴ plaque-forming units (PFU) of rAAV.

In certain embodiments, vector stock is produced by one or more post processing steps, such as filtration and/or concentration (e.g., depth filtration, dead-end filtration, tangential flow filtration (TFF), and diafiltration), multi-column chromatography purification, final concentration/buffer exchange, etc., to obtain vector stocks with sufficient purity for administration to animals, including human. In certain embodiments, the purification process and the purified vector stock satisfy GMP standard.

In certain embodiments, the titer of the rAAV vector stocks is about 1-2×10⁷ PFU/ml, about 1-2×10⁸ PFU/ml, about 1-2×10⁹ PFU/ml, about 1-2×10¹⁰ PFU/ml, about 1-2×10¹¹ PFU/ml, or about 1-2×10¹² PFU/ml.

In certain embodiments, the total yield of the rAAV vector stock is about 1-25×10¹⁴ total VG of purified rAAV, about 1-10×10¹⁴ total VG of purified rAAV, about 1-5×10¹⁴ total VG of purified rAAV, or about 2-4×10¹⁴ total VG of purified rAAV.

In certain embodiments, rAAV vectors so produced are further purified from crude cell lysates by ion-exchange chromatography and/or by iodixanol density gradient centrifugation to ensure high final product purity. In certain embodiments, rAAV vectors so produced qualify as a clinical-grade vector batch.

In certain embodiments, the AAV production method of the invention further comprises determining the titer, purity, and/or potency of the rAAV vectors so produced.

This may include characterizing the purified rAAV stocks using, e.g., silver staining of SDS-PAGE separation of proteins to determine purity.

### 7. Treatment of Muscular Dystrophy Using AAV

The subject single vector-based system can be used for large scale production of rAAV, which in turn can be used in gene therapy for treating various forms of muscular dystrophy, such as Duchenne's muscular dystrophy (DMD), Myotonic dystrophy, Becker muscular dystrophy (BMD), Limb-girdle muscular dystrophy (LGMD), Facioscapulohumeral muscular dystrophy (FSH), Congenital muscular dystrophy (CDM), Oculopharyngeal muscular dystrophy (OPMD), Distal muscular dystrophy, Emery-Dreifuss muscular dystrophy (EDMD), etc. In certain embodiments, the muscular dystrophy is DMD or BMD.

Thus another aspect of the invention provides a method of treating muscular dystrophy (such as DMD and BMD) in a subject in need thereof, the method comprising administering to the subject a therapeutically effective amount of a recombinant AAV (rAAV) vector encoding a functional version of the gene defective in the muscular dystrophy, such as a microdystrophin gene, wherein the rAAV is produced by the method of the invention using the subject single vector and complementary system.

In certain embodiments, the microdystrophin gene is one described in U.S. Pat. Nos. 7,906,111; 7,001,761; 7,510,867; 6,869,777; 8,501,920; 7,892,824; WO2020/086844; PCT/US2016/013733; or U.S. Pat. No. 10,166,272 (all incorporated herein by reference).

In certain embodiments, the microdystrophin gene comprises a coding sequence for the R1, R16, R17, R23, and R24 spectrin-like repeats of the full-length dystrophin protein (such as one described in PCT/US2016/013733).

In certain embodiments, the method further comprises producing the rAAV by the method of the invention using the subject single vector system, prior to administering to the subject the rAAV so produced.

### EXAMPLES

The examples described herein, including all exemplary plasmids and sequence elements thereof described herein, and as represented in FIGS. 1A and 1B, such as specific ITR sequences, specific gene of interest (such as microdystrophin), specific Rep/Cap, specific promoter (such as CK8), specific polyA sequence, etc., are for illustrative purpose only, and are not limiting in any respect.

### Example 1 AAV Production Using Single Plasmid System

### Single Plasmid Generation, Transfection and Sampling

Exemplary plasmids, including the exemplary plasmid depicted in FIGS. 1A and 1B, were generated by ligation and gel-purification. Then the plasmid was transformed into bacteria, and plasmid sequences were verified by next-generation sequencing.

Single plasmids comprising a microdystrophin variant as the gene of interest (GOI), Rep/Cap genes and Helper genes, wherein the Rep/Cap genes and Helper genes have the same direction of transcription as depicted in FIGS. 1A and 1B, were generated (OPT). Single plasmids comprising the same component but with the Rep/Cap genes and Helper genes having different direction of transcription (OPT-revHelp) were also generated for testing.

Single plasmids were transfected into VP2 cells using FectoVIR®-AAV transfection reagents according to the manufacturer's recommendation. Viral titre collected from the supernatant and cells after transfection with the single plasmids were compared to viral titre produced from transfection with a dual plasmid system, in which the GOI and Rep/Cap cassettes are on the same plasmid and the Helper genes are on a separate plasmid.

### AAV2 Production Using Single Plasmid Transfection System

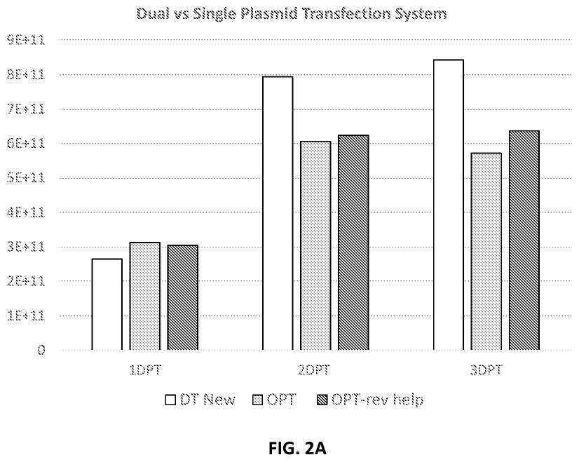

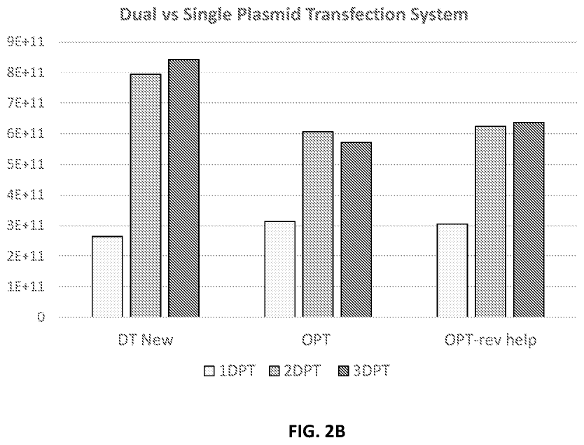

Results from transfecting VP2 cells with the single plasmid sytems or dual plasmid system are depicted in FIGS. 2A-2B.

As shown in FIGS. 2A-2B, despite their very large size (~20.6 kb), single plasmids comprising both Rep/Cap, GOI and Helper sequences with either Rep/Cap-Helper orientation were suprisingly sufficient at producing AAV particles.

1. A recombinant DNA vector comprising:
(a) a gene-of-interest (GOI) flanked by a 5′ adeno-associated virus (AAV) ITR (such as AAV2 5′ ITR) sequence and a 3′ ITR sequence (such as AAV2 3′ ITR);
(b) a coding sequence for an AAV Rep (such as AAV2 Rep, or Rep2) compatible with said 5′ AAV ITR and/or said 3′ AAV ITR;
(c) a coding sequence for an AAV Cap; and
(d) a coding sequence for AAV Helper genes sufficient for enabling AAV packaging;
wherein the coding sequence for the AAV Rep and the coding sequence for the AAV Cap are within a RepCap cassette comprising an operably-linked RepCap promoter (such as the AAV P5 promoter); and,
wherein the GOI flanked by the 5′ and 3′ ITRs is positioned upstream of (e.g., adjacent to, or immediately 5′ to) the 5′ end of the promoter of the RepCap cassette.

2. The recombinant DNA vector of claim 1, wherein expression of said AAV Rep, Cap and Helper genes in a host cell comprising AAV helper genes is sufficient to package an AAV vector genome (vg) comprising the GOI flanked by the 5′- and 3-ITR sequences into an AAV capsid comprising said AAV Cap.

3. The recombinant vector of any one of claims 1-2, wherein the recombinant vector is in a plasmid.

4. The recombinant vector of any of claims 1-3, wherein the GOI is within a pro-AAV cassette comprising the GOI operably linked to a promoter.

5. The vector of claim 4, wherein the pro-AAV cassette further comprises:
(1) an enhancer that promotes the transcription of the GOI from the promoter;
(2) a 5′ UTR;
(3) a Kozak sequence;
(4) a heterologous intron that promotes transcription and/or translation of the GOI;
(5) a 3′ UTR;
(6) a WPRE sequence; and/or
(7) a polyA signal sequence.

6. The recombinant vector of any one of claims 4-5, wherein the RepCap cassette and the pro-AAV cassette:
i) are immediately adjacent to each other (e.g., with substantially no intervening polynucleotide sequence);
ii) are not immediately adjacent to each other;
iii) have the same transcription direction;
iv) having opposite transcription direction.

7. The recombinant vector of claim 6, further comprising a bacterial replication Ori gene, a selection marker (such as an antibiotic resistance gene, e.g., Kan^{R} or Amp^{R}) under the transcriptional control of a selection marker promoter.

8. The recombinant vector of any one of claims 1-7, wherein said GOI is a functional equivalent of dystrophin (e.g., a dystrophin minigene encoding a functional micro-dystrophin protein).

9. The recombinant vector of any one of claims 1-8, wherein said GOI includes a gene responsible for/defective in LGMD2E (limb-girdle muscular dystrophy type 2E), LGMD2D (limb-girdle muscular dystrophy type 2D), LGMD2C (limb-girdle muscular dystrophy type 2C), LGMD2B (limb-girdle muscular dystrophy type 2B), LGMD2L (limb-girdle muscular dystrophy type 2L), LGMD2I (limb-girdle muscular dystrophy type 2I), or a gene or coding sequence for NAGLU (α-N-acetylglucosaminidase, for Sanfilippo syndrome or mucopolysaccharidosis type IIIB (MPS IIIB)), sulfamidase or SGSH (for mucopolysaccharidosis type IIIA or MPS IIIA), Factor IX, Factor VIII, Myotubularin 1 (MTM1), Survival of Motor Neuron (SMN, for spinal muscular atrophy or SMA), GalNAc transferase GALGT2, calpain-3(CAPN-3), acid alpha-glucosidase (GAA, for Pompe disease), alpha-galactosidase A or GLA (for Fabry disease), glucocerebrosidase, dystrophin or microdystrophin.

10. The recombinant vector of any one of claims 1-8, wherein the GOI is a microdystrophin gene (e.g., one described in U.S. Pat. Nos. 7,906,111; 7,001,761; 7,510,867; 6,869,777; 8,501,920; 7,892,824; PCT/US2016/013733; or U.S. Pat. No. 10,166,272).

11. The recombinant vector of claim 10, wherein the microdystrophin gene comprises a coding sequence for R16 and R17 spectrin-like repeats for the full length dystrophin protein (such as one described in U.S. Pat. No. 7,892,824).

12. The recombinant vector of claim 11, wherein the microdystrophin gene comprises a coding sequence for the R1, R16, R17, R23, and R24 spectrin-like repeats of the full-length dystrophin protein (such as the microdystrophin gene described in PCT/US2016/013733).

13. The recombinant vector of any one of claims 1-12, wherein said 5′ and 3′ AAV ITR sequences flanking said GOI are both from AAV1, AAV2, AAV3A, AAV3B, AAV4, AAV5, AAV6, AAV7, AAV8, AAV9, AAV10, AAV11, AAV12, AAV13, or AAV-DJ.

14. The recombinant vector of claim 13, wherein the tropism of the AAV include skeletal muscle (such as AAV1, AAV6, AAV7, AAV8, AAV9, or a derivative thereof, preferably AAV9 or a derivative thereof such as SLB-101 described in WO 2021/072197).

15. The recombinant vector of any one of claims 1-14, wherein said AAV ITR, said AAV Rep, and said AAV Cap are from the same or different AAVs.

16. The recombinant vector of claim 15, wherein said AAV ITR is AAV2 ITR, said AAV Rep is Rep2 from AAV2, and said AAV Cap is Cap9 from AAV9 or a derivative thereof (such as SLB-101 described in WO 2021/072197).

17. The recombinant vector of any one of claims 1-16, wherein said coding sequence for AAV Rep and Cap proteins is under the transcriptional control of a promoter, such as an AAV p5 promoter, an upstream HSV promoter, a modified p5 promoter lacking RBE (Rep-Binding Element), or a ubiquitous promoter (such as CMV promoter, EF1a promoter, CAG promoter, CB promoter etc).

18. The recombinant vector of claim 16, wherein the AAV RepCap promoter is an AAV p5 promoter.

19. The recombinant vector of any one of claims 1-18, wherein said AAV helper genes comprise adenoviral, herpesviral, or papillomaviral genes for AAV packaging (such as E1A, E1B, E2A, E4 and VA RNA), optionally operably linked to a promoter as one transcriptional unit.

20. A plasmid comprising the recombinant vector of any one of claims 1-19.

21. A composition or a kit, comprising the vector of any one of claims 1-19 or the plasmid of claim 20.

22. A host cell comprising the recombinant vector of any one of claims 1-19, or the plasmid system of claim 21.

23. The host cell of claim 22, which is a HEK293 cell (such as an Expi293F cell), a HeLa cell, an A549 cell, a BHK cell, a VP2 cell, or an insect cell (such as Sf9).

24. A method of propagating/amplifying/producing a recombinant replication-defective AAV viral particle encapsidating the GOI of any one of claims 1-19, the method comprising: introducing the vector of any one of claims 1-19 or the plasmid of claim 20 into a host cell.

25. The method of claim 24, further comprising harvesting the recombinant replication-defective AAV viral particle from the host cell.

26. The method of claim 24 or 25, wherein the host cell is a HEK293 cell (such as an Expi293F cell), a HeLa cell, an A549 cell, a BHK cell, a VP2 cell or an insect cell (such as Sf9).

27. The method of any one of claims 24-26, wherein the recombinant vector or plasmid is introduced to the host cell by transient transfection.
